(19) United States
(12) Patent Application Publication
(10) Pub. No.: US 2020/0306140 A1
(43) Pub. Date: Oct. 1, 2020

(54) Medication and Medical Item Storage Container

(71) Applicant: Kwik-Care, LLC, Hughesville, PA (US)

(72) Inventor: Laurie Fontaine, Hughesville, PA (US)

(21) Appl. No.: 16/832,643

(22) Filed: Mar. 27, 2020

Related U.S. Application Data
(60) Provisional application No. 62/826,170, filed on Mar. 29, 2019.

(51) Int. Cl. A61J 7/00; A61F 17/00; A45C 11/00; G09B 19/00

(52) U.S. Cl. CPC A61J 7/0069; A61F 17/00; A45C 11/00; A45C 2011/007; A61J 2205/20; A61J 2205/30; G09B 19/00

(57) ABSTRACT

A storage container for medication and medical items includes a first section defining a first cavity to receive a first portion of the medication and medical items; a second section defining a second cavity to receive a second portion of the medication and medical items; at least one tray positioned in at least one of the first section and the second section configured to hold the respective medication and medical items; and at least one educational information card positioned in at least one of the first section and the second section. The educational information card includes information pertaining to the medication and how to use the medical items held in the at least one tray.

### CROSS REFERENCE TO RELATED APPLICATION

This application claims the benefit of U.S. Provisional Patent Application No. 62/826,170, entitled “Medication and Medical Item Storage Container,” filed Mar. 29, 2019, the disclosure of which is incorporated in its entirety by reference.

### BACKGROUND OF THE INVENTION

### Field of the Invention

The present disclosure is generally directed to a storage container for medication and medical items and, in particular, to a storage container for holding different types of medication and medical items with a corresponding educational information card that includes information pertaining to the medication and how to use the medical items held in the storage container.

### Description of Related Art

Consumers purchase multiple, unnecessary, and occasionally inappropriate or non-applicable over-the-counter medications and ancillary items when faced with minor acute illnesses and/or injuries. The average consumer does not usually know the correct items to purchase and/or how to appropriately treat symptoms that can be addressed with over-the-counter treatment items and medications. Presently, consumers are influenced (sometimes inappropriately) by advertising on over-the-counter items and medications to treat limited aspects of an illness or injury.

The limitations of current solutions to this problem are that there is nothing available that provides consumers with a comprehensive approach to: 1) treating symptoms, 2) using condition-specific items and/or medications, and 3) achieving the best health outcome. Even in the event a consumer is able to schedule an appointment with a primary care provider to address his/her health issues, the present-day primary care provider does not have the time in their daily schedule to recite all of these elements and information in detail, and provide specific, written guidance, as needed by each individual patient for individual conditions.

### SUMMARY OF THE INVENTION

In view of the foregoing deficiencies with current products, there is a current need in the art for a medication and medical item storage container that holds each of these items in an easy-to-use and easily identifiable manner to ensure a user is properly using the items to achieve improved results in addressing the individual's health issue.

In one example of the present disclosure, a storage container for medication and medical items includes a first section defining a first cavity to receive a first portion of the medication and medical items; a second section defining a second cavity to receive a second portion of the medication and medical items; at least one tray positioned in at least one of the first section and the second section configured to hold the respective medication and medical items; and at least one educational information card positioned in at least one of the first section and the second section, wherein the educational information card includes information pertaining to the medication and how to use the medical items held in the at least one tray.

The at least one tray may include a first tray and a second tray positioned in the first section and the second section, respectively, each configured to hold the respective medication and medical items of each tray.

The at least one educational information card may include a first educational information card and a second educational information card positioned in the first section and the second section, respectively, wherein each educational information card includes information pertaining to the respective medication and how to use the respective medical items held in each tray.

Each of the first educational information card and the second educational information card may include a foldout including a plurality of educational information displays, wherein the foldout has a hinged connection with each tray.

Each of the first educational information card and the second educational information card may be configured to form a lid for each of the first tray and the second tray, respectively.

The at least one tray may include medication and medical items pertaining to a designated condition.

The information pertaining to the medication and how to use the medical items pertaining to a designated condition may include condition-specific education configured to optimize a health outcome.

The first tray and the second tray positioned in the first section and the second section, respectively, each may include medication and medical items pertaining to a designated condition.

The information pertaining to the medication and how to use the medical items pertaining to a designated condition of the first tray and the second tray may include condition-specific education configured to optimize a health outcome.

The first section and the second section may be connected by a hinge and may be configured to be fastened together by a fastening member.

The storage container may include an informational sleeve surrounding the storage container, wherein the informational sleeve includes information pertaining to the medication and how to use the medical items held in the storage container.

The at least one educational information card may include at least one of: content pictures describing each medication and/or medical item, educational elements explaining reason for use for each medical item, and application education for each medication and/or how to use each medical item.

In another example of the present disclosure, a storage container for medication and medical items includes a first section defining a cavity to receive a first portion of the medication and medical items; a second section defining a second cavity to receive a second portion of the medication and medical items; at least one divider positioned in at least one of the first section and the second section to create individual spaces to hold the medication and medical items; and an educational information card positioned in the storage container, wherein the educational information card includes information pertaining to the medication and how to use the medical items held in the storage container.

The storage container may include medication and medical items pertaining to a designated condition.

The information pertaining to the medication and how to use the medical items pertaining to a designated condition may include condition-specific education configured to optimize a health outcome.

The storage container may include at least one cover panel positioned over the at least one divider.

The at least one cover panel may include a first portion and a second portion, wherein the first portion and the second portion are removably connected to one another.

The educational information card may include a first side and a second side, wherein the first side and the second side of the educational information card includes information pertaining to the medication and how to use the medical items held in the first section and the second section, respectively, of the storage container.

The educational information card may be fixedly attached to the storage container.

The educational information card may be removably positioned within the storage container.

Various aspects of the present disclosure may be further characterized by one or more of the following clauses:

Clause 1: A storage container for medication and medical items, comprising: a first section defining a first cavity to receive a first portion of the medication and medical items; a second section defining a second cavity to receive a second portion of the medication and medical items; at least one tray positioned in at least one of the first section and the second section configured to hold the respective medication and medical items; and at least one educational information card positioned in at least one of the first section and the second section, wherein the educational information card includes information pertaining to the medication and how to use the medical items held in the at least one tray.

Clause 2: The storage container of Clause 1, wherein the at least one tray comprises: a first tray and a second tray positioned in the first section and the second section, respectively, each configured to hold the respective medication and medical items of each tray.

Clause 3: The storage container of any of Clauses 1 and 2, wherein the at least one educational information card comprises: a first educational information card and a second educational information card positioned in the first section and the second section, respectively, wherein each educational information card includes information pertaining to the respective medication and how to use the respective medical items held in each tray.

Clause 4: The storage container of Clause 3, wherein each of the first educational information card and the second educational information card comprises: a foldout including a plurality of educational information displays, wherein the foldout has a hinged connection with each tray.

Clause 5: The storage container of any of Clauses 3 and 4, wherein each of the first educational information card and the second educational information card is configured to form a lid for each of the first tray and the second tray, respectively.

Clause 6: The storage container of any of Clauses 1-5, wherein the at least one tray includes medication and medical items pertaining to a designated condition.

Clause 7: The storage container of Clause 6, wherein the information pertaining to the medication and how to use the medical items pertaining to a designated condition includes condition-specific education configured to optimize a health outcome.

Clause 8: The storage container of Clause 2, wherein the first tray and the second tray positioned in the first section and the second section, respectively, each includes medication and medical items pertaining to a designated condition.

Clause 9: The storage container of Clause 8, wherein the information pertaining to the medication and how to use the medical items pertaining to a designated condition includes condition-specific education configured to optimize a health outcome.

Clause 10: The storage container of any of Clauses 1-9, wherein the first section and the second section are connected by a hinge and are configured to be fastened together by a fastening member.

Clause 11: The storage container of any of Clauses 1-10, further comprising: an informational sleeve surrounding the storage container, wherein the informational sleeve includes information pertaining to the medication and how to use the medical items held in the storage container.

Clause 12: The storage container of any of Clauses 1-11, wherein the at least one educational information card includes at least one of: content pictures describing each medication and/or medical item, educational elements explaining reason for use for each medical item, and application education for each medication and/or how to use each medical item.

Clause 13: A storage container for medication and medical items, comprising: a first section defining a cavity to receive a first portion of the medication and medical items; a second section defining a second cavity to receive a second portion of the medication and medical items; at least one divider positioned in at least one of the first section and the second section to create individual spaces to hold the medication and medical items; and an educational information card positioned in the storage container, wherein the educational information card includes information pertaining to the medication and how to use the medical items held in the storage container.

Clause 14: The storage container of Clause 13, wherein the storage container includes medication and medical items pertaining to a designated condition.

Clause 15: The storage container of Clause 14, wherein the information pertaining to the medication and how to use the medical items pertaining to a designated condition includes condition-specific education configured to optimize a health outcome.

Clause 16: The storage container of any of Clauses 13-15, further comprising: at least one cover panel positioned over the at least one divider.

Clause 17: The storage container of Clause 16, wherein the at least one cover panel comprises a first portion and a second portion, wherein the first portion and the second portion are removably connected to one another.

Clause 18: The storage container of any of Clauses 13-17, wherein the educational information card comprises a first side and a second side, wherein the first side and the second side of the educational information card includes information pertaining to the medication and how to use the medical items held in the first section and the second section, respectively, of the storage container.

Clause 19: The storage container of any of Clauses 13-18, wherein the educational information card is fixedly attached to the storage container.

Clause 20: The storage container of any of Clauses 13-19, wherein the educational information card is removably positioned within the storage container.

### BRIEF DESCRIPTION OF THE DRAWINGS

FIG. 1 is a perspective view of a storage container according to one example of the present disclosure;

FIG. 2 is another perspective view of the storage container of FIG. 1 showing a foldout including a plurality of educational information displays;

FIG. 3 is a perspective view of a storage container according to another example of the present disclosure being illustrated with a cover panel in an open position;

FIG. 4 is another perspective view of the storage container of FIG. 3 with the cover panel in a closed position;

FIG. 5 is a perspective view of a divider used in the storage container of FIG. 3 and FIG. 4;

FIG. 6 is a perspective view of a dividing wall used in the divider of FIG. 5;

FIG. 7 is a perspective view of the storage container of FIGS. 1-4 showing an informational sleeve surrounding the storage container; and

FIG. 8 is a perspective view of a storage container according to another example of the present disclosure.

Like reference symbols in the various drawings indicate like elements.

### DESCRIPTION OF THE INVENTION

For purposes of the description hereinafter, spatial orientation terms, as used, shall relate to the referenced embodiment as it is oriented in the accompanying drawings, figures, or otherwise described in the following detailed description. However, it is to be understood that the embodiments described hereinafter may assume many alternative variations and configurations. It is also to be understood that the specific components, devices, features, and operational sequences illustrated in the accompanying drawings, figures, or otherwise described herein are simply exemplary and should not be considered as limiting.

As used in the specification, the singular form of “a”, “an”, and “the” include plural referents unless the context clearly dictates otherwise.

As used in the claims or the specification, the language “at least one of X, Y, and Z” means “only X, only Y, or only Z; at least one of X and at least one of Y, at least one of X and at least one of Z, or at least one of Y and at least one of Z; or at least one of X and at least one of Y and at least one of Z.”

The present disclosure is generally directed to a storage container for medication and medical items and, in particular, to a storage container for holding different types of medication and medical items with a corresponding educational information card that includes information pertaining to the medication and how to use the medical items held in the storage container.

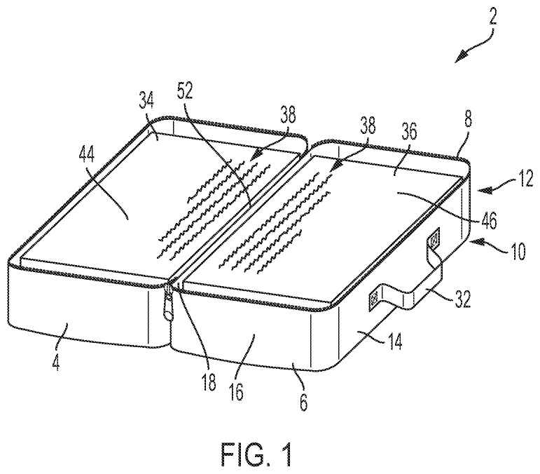

Certain preferred and non-limiting aspects of the components of the storage container are illustrated in FIGS. 1-8.

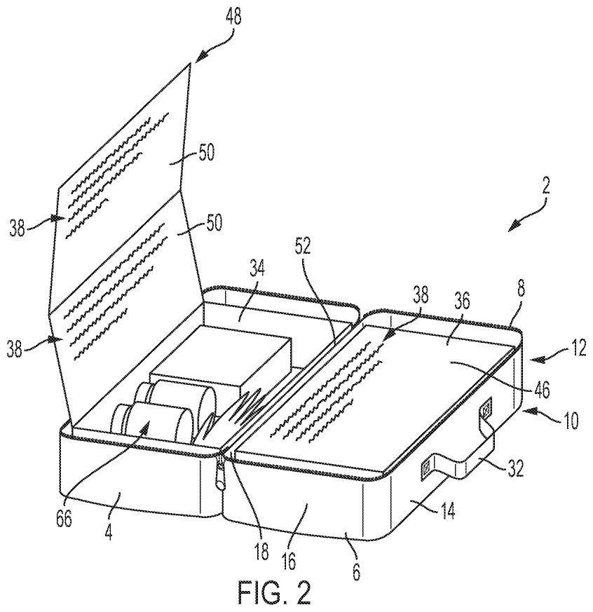

With reference to FIG. 1 and FIG. 2, a first example of a storage container 2 according to the present disclosure is shown and described. The storage container 2 may include a first section 4 and a second section 6 that can be fastened to one another to form the storage container 2. In particular, the first section 4 and the second section 6 may be connected to one another via a hinge 52 and may be fastened to one another using a fastening member 8. In one example, the fastening member 8 is a zipper, but it is also contemplated that the fastening member 8 may be buttons, snaps, laces, or any other suitable fastening member that connects the first section 4 to the second section 6. Each section 4, 6, includes a bottom panel 10, and four side panels 12, 14, 16, 18. The bottom panel 10 and the side panels 12, 14, 16, 18 of each section 4, 6 define a cavity therein for holding medication and medical items 66 (medication and medical items 66 not shown in FIG. 1). In one example, the storage container 2 is configured to hold many different types of medications, including over-the-counter medications, and smaller medical items and items, including gauze pads, bandages, medical grade gloves, cleaning agents, disinfectants, and other medical items that may be needed to address common health issues. It is to be understood that this list of medication and medical items 66 is not meant to be restrictive, but instead is meant to include any medical-related items that one may need to address a health issue.

The storage container may include at least one tray 34, 36 positioned in at least one of the first section 4 and the second section 6. It is also contemplated that the storage container may include at least one tray 34, 36 in just one of the first section 4 and the second section 6. The at least one tray may include a first tray 34 and a second tray 36 positioned in the first section 4 and the second section 6, respectively. Each of the first tray 34 and the second tray 36 may be configured to hold the respective medication and medical items 66 of each tray. The at least one tray 34, 36 may be removably inserted into each section 4, 6 so that the at least one tray 34, 36 can be removed in the event a large medication or medical device or item is stored in the section 4, 6. To ensure that no medications or medical items are contaminated, the at least one tray 34, 36 may be shrink wrapped prior to final packaging within the storage container 2.

With continued reference to FIG. 1 and FIG. 2, at least one educational information card 44, 46 is positioned in at least one of the first section 4 and the second section 6. It is also contemplated that at least one educational information card 44, 46 is positioned in just one of the first section 4 and the second section 6. The educational information card 44, 46 includes information 38 pertaining to the medication and how to use the medical items held in the at least one tray 34, 36. The at least one educational information card 44, 46 may include a first educational information card 44 and a second educational information card 46 positioned in the first section 4 and the second section 6, respectively. Each of the first educational information card 44 and the second educational information card 46 may include information 38 pertaining to the medication and how to use the medical items held in each tray. Each of the first educational information card 44 and the second educational information card 46 may be configured to form a lid for each of the first tray 34 and the second tray 36, respectively.

With reference to FIG. 2, each of the first educational information card 44 and the second educational information card 46 may include a foldout 48. The foldout 48 may include a plurality of educational information displays 50, wherein the foldout 48 has a hinged connection with each tray. The educational information displays 50 may include information 38 pertaining to the medication and how to use the medical items held in each tray. The foldout 48 may be configured to form a lid for each of the first tray 34 and the second tray 36, respectively. The at least one tray 34, 36 may include medication and medical items 66.

With continued reference to FIG. 1 and FIG. 2, the at least one tray 34, 36 may include medication and medical items 66 pertaining to a designated condition. The information 38 pertaining to the medication and how to use the medical items pertaining to a designated condition may include condition-specific education configured to optimize a health outcome. Additionally, first tray 34 and the second tray 36 positioned in the first section 4 and the second section 6, respectively, may include medication and medical items 66 pertaining to a designated condition. The information 38 pertaining to the medication and how to use the medical items pertaining to a designated condition of the first tray 34 and the second tray 36 may include condition-specific education configured to optimize a health outcome.

The educational information card 44, 46 may include content pictures describing each medication and/or medical item, educational elements explaining reason for use for each medical item, and application education for each medication and/or how to use each medical item. The educational information card 44, 46 may also include information pertaining to the specific medication and medical item 66 that are held in the storage container, content pictures indicating in which tray 34, 36 each medication and/or medical item is stored/to be used, and educational elements explaining reason for use. The educational information card 44, 46 may also include the best use of the items held in the storage container 2 for the best health outcomes for the designated illness and/or injury for that specific storage container 2 and/or tray 34, 36. The educational information card 44, 46, however, does not give directed medical advice nor does it lead a user to make a diagnosis regarding an illness or injury.

The educational information card 44, 46 may include easy-to-read large font information and may be laminated to prevent the educational information card 44, 46 from becoming wet or damaged. In another example, the educational information card 44, 46 may be held in a sleeve attached to the storage container 2 that permits different educational information cards to be removably inserted therein according to the specific illness or injury that is to be addressed with the items held in the storage container 2. In another example, the educational information card 44, 46 may include picture-code labelling that corresponds to the contents of the storage container 2. In another example, the educational information card 44, 46 may include color-code labelling that corresponds to color-code labelling of the trays 34, 36 so that users that cannot read are able to identify which items in the storage container 2 should be used addressing the symptoms. In one example, each section 4, 6 includes a handle 32 to assist the user in carrying the storage container 2. It is also contemplated that just one section 4, 6 includes a handle 32 to assist the user in carrying the storage container 2. It is also contemplated that an internal carabiner may be provided on the storage container 2 for attachment to a backpack or belt loop, if desired.

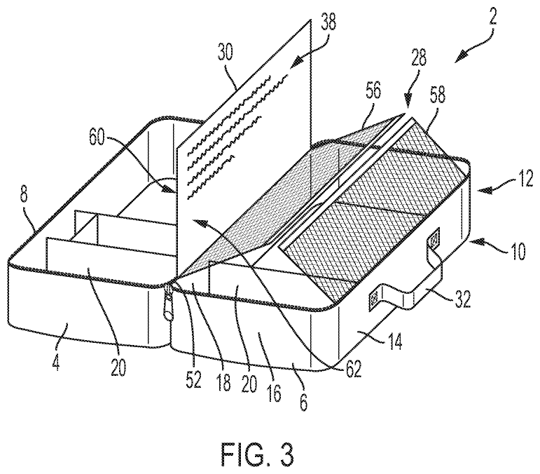

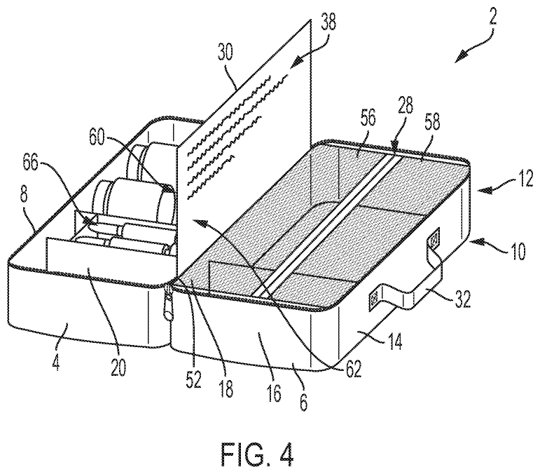

With reference to FIG. 3 and FIG. 4, a second example of a storage container 2 according to the present disclosure is shown and described. The storage container 2 may include a first section 4 and a second section 6 that can be fastened to one another to form the storage container 2. In particular, the first section 4 and the second section 6 may be connected to one another via a hinge 52 and may be fastened to one another using a fastening member 8. In one example, the fastening member 8 is a zipper, but it is also contemplated that the fastening member 8 may be buttons, snaps, laces, or any other suitable fastening member that connects the first section 4 to the second section 6. Each section 4, 6, includes a bottom panel 10, and four side panels 12, 14, 16, 18. The bottom panel 10 and the side panels 12, 14, 16, 18 of each section 4, 6 define a cavity therein for holding medication and medical items 66 (medication and medical items 66 not shown in FIG. 3). In one example, the storage container 2 is configured to hold many different types of medications, including over-the-counter medications, and smaller medical items and items, including gauze pads, bandages, syringes, medical grade gloves, cleaning agents, disinfectants, and other medical items that may be needed to address common health issues. It is to be understood that this list of medication and medical items 66 is not meant to be restrictive, but instead is meant to include any medical-related items that one may need to address a health issue.

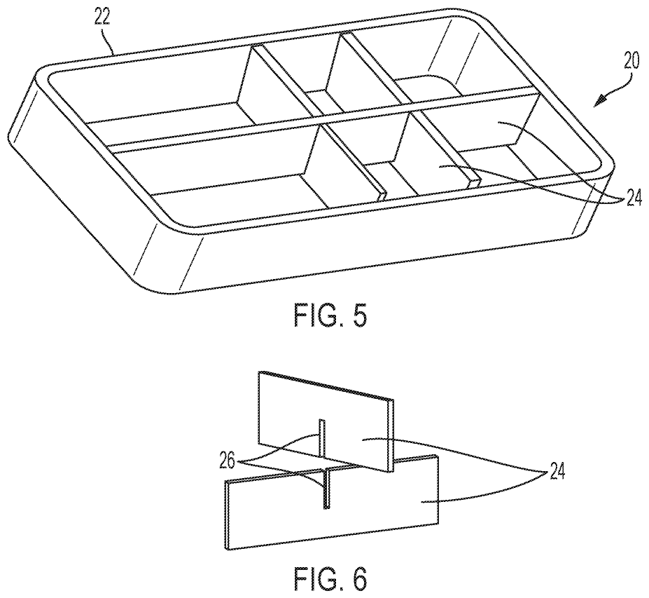

Each section 4, 6 of the storage container 2 may include a divider 20 that is used to create individual spaces within each section 4, 6 to hold the variety of medication and medical items 66 that are provided in the storage container 2. The divider 20 may be removably inserted into each section 4, 6 so that the divider 20 can be removed in the event a large medication or medical device or item is stored in the section 4, 6. Otherwise, as shown in FIG. 5 and FIG. 6, the divider 20 includes an outer frame 22 and a plurality of inner dividing members 24 that create the individual spaces within the divider 20. The dividing members 24 may be adjustable so that a user can adjust the size of each of the spaces within the divider 20 to accommodate specific items that are being held in the divider 20. With reference to FIG. 6, the dividing members 24 may include slots 26 that can receive a portion of another dividing member 24 to removably connect the dividing members 24 together. Therefore, each dividing member 24 can be arranged and positioned so that the user can create storage spaces that accommodate the items held in the storage container 2. The divider 20 may be made of cardboard, plastic, medical-grade plastic, or any other suitable material that can be used to maintain a frigid framework within the storage container 2.

With continued reference to FIG. 3 and FIG. 4, each section 4, 6 may have a cover panel 28 that is provided to create a covering over the divider 20 so that the medication and medical items 66 held in each section 4, 6 to not fall out of the storage container 2 after the storage container 2 has been opened. It is also contemplated that only one of the sections 4, 6 may have a cover panel 28. The cover panel 28 may also assist in maintaining the sterility and cleanliness of the inner cavities of the sections 4, 6 to ensure that no medications or medical items are contaminated. In one example, the cover panel 28 may be split into a first portion 56 and a second portion 58 so that the cover panel 28 can be moved between an open position (FIG. 3) in which the cover panel 28 portions are separated from one another so that the user can access the items held in the specific section 4, 6 of the storage container 2, and a closed position (FIG. 4) in which the cover panel 28 portions are held together to prevent items in the section 4, 6 falling out of the storage container 2. The first portion 56 and the second portion 58, thus, can be removably connected to one another. The cover panel 28 may be made of a mesh material to permit air flow through the storage container 2 and permit a user to view the items that are held in each section 4, 6 of the storage container 2. It is also contemplated, however, that other materials may be used for the cover panel 28, as desired. The first portion 56 and a second portion 58 of the cover panel 28 may be removably connected to one another using a zipper, buttons, snaps, hook and loop fasteners, or any similar mechanisms.

With continued reference to FIG. 3 and FIG. 4, an educational information card 30 may be positioned between the sections 4, 6 of the storage container 2. The educational information card may include a first side 60 and a second side 62. The first side 60 and the second side 62 of the educational information card may include information 38 pertaining to the medication and how to use the medical items held in the first section 4 and the second section 6, respectively, of the storage container. The educational information card 30 may be fixedly attached to the storage container 2 or may be removably positioned within the storage container 2 between the sections 4, 6 so that the user can remove the educational information card 30 from the storage container 2.

The educational information card 30 may include information pertaining to the specific medication and medical items 66 that are held in the storage container, content pictures indicating in which storage space each medication and/or medical item is stored/to be used, educational elements explaining reason for use, and/or application education for the medications and/or medical items. The educational information card 30 may also include the best use of the items held in the storage container 2 for the best health outcomes for the designated illness and/or injury for that specific storage container 2. The educational information card 30, however, does not give directed medical advice nor does it lead a user to make a diagnosis regarding an illness or injury.

The educational information card 30 may also include easy-to-read large font information and may be laminated to prevent the educational information card 30 from becoming wet or damaged. In another example, the educational information card 30 may be held in a sleeve attached to the storage container 2 that permits different educational information cards to be removably inserted therein according to the specific illness or injury that is to be addressed with the items held in the storage container 2. In another example, the educational information card 30 may include picture-code labelling that corresponds to the contents of the storage container 2. In another example, the educational information card 30 may include color-code labelling that corresponds to color-coded labelling the divider 20 so that users that cannot read can easily identify which items in the storage container 2 should be used during the specific steps of condition-specific education. In one example, each section 4, 6 includes a handle 32 to assist the user in carrying the storage container 2. It is also contemplated that just one section 4, 6 includes a handle 32 to assist the user in carrying the storage container 2. It is also contemplated that an internal carabiner may be provided on the storage container 2 for attachment to a backpack or belt loop, if desired.

The storage container 2 is a comprehensive care kit that is designed to provide the general public with over-the-counter medication and medical items, as well as easy-to-understand education to self-address minor, acute illnesses and injuries. In some non-limiting embodiments and aspects, each storage container 2 can contain multiple items to address the illness or injury designated by the specific storage container 2 for a period of 7-10 days. In some non-limiting embodiments and aspects, each storage container 2 can contain multiple items to address the illness or injury designated by the specific storage container 2 for a period of 3-5 days. It is also contemplated that more or less items may be held in the storage container 2 in the event less or more days are required to address the illness or injury. The storage container 2 provides the user with multiple, comprehensive items to address the illness or injury that would most likely not be purchased individually due to high over-the-counter costs and large package quantities that are often note utilized within a normal acute illness or injury course.

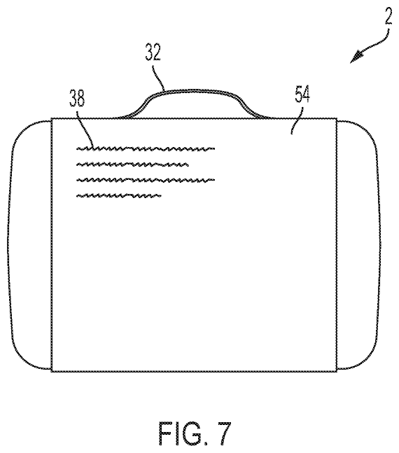

With reference to FIG. 7, each storage container 2 may include an informational sleeve 54 surrounding the storage container 2. In one example, the informational sleeve 54 includes information 38 pertaining to the medication and how to use the medical items held in the storage container. In another example, the informational sleeve 54 may include a cardboard sleeve, that indicates the specific illness and/or injury that is to be addressed with the items held in the storage container 2. An intact cardboard sleeve at the time of purchase for the user helps to assure quality control by limiting any attempts at tampering with the items held within the storage container 2. Additionally, each specific storage container 2 may include a color-coded indicator thereon to indicate the type of items held in the storage container 2. The color-coded indicator may be a zip tie that is inserted through the two zippers that open and close the storage container 2. An intact zip tie also helps to assure quality control by limiting any attempts at tampering with internal items prior to purchase.

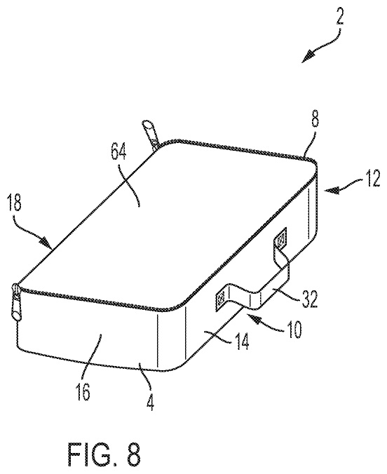

With reference to FIG. 8, a third example of a storage container 2 according to the present disclosure is shown and described. The storage container 2 may include a first section 4 and a cover flap 64 that can be fastened to one another to form the storage container 2. In particular, the first section 4 and the cover flap 64 may be fastened to one another using a fastening member 8. In one example, the fastening member 8 is a zipper, but it is also contemplated that the fastening member 8 may be buttons, snaps, laces, or any other suitable fastening member that connects the first section 4 to the cover flap 64. The first section 4 includes a bottom panel 10, and four side panels 12, 14, 16, 18. The bottom panel 10 and the side panels 12, 14, 16, 18 of the first section 4 define a cavity therein for holding medication and medical items 66. In one example, the storage container 2 is configured to hold many different types of medications, including over-the-counter medications, and smaller medical items and items, including gauze pads, bandages, medical grade gloves, cleaning agents, disinfectants, and other medical items that may be needed to address common health issues. It is to be understood that this list of medication and medical items 66 is not meant to be restrictive, but instead is meant to include any medical-related items that one may need to address a health issue.

Although not shown in FIG. 8, at least one educational information card can be positioned in the first section 4 of the storage container 2. The educational information card can include information pertaining to the medication and how to use the medical items held in the storage container 2. The educational information card may be removably positioned within the storage container.

With continued reference to FIG. 8, in some non-limiting embodiments and aspects, each storage container 2 can contain multiple items to address the illness or injury designated by the specific storage container 2 for a period of 3-5 days. It is also contemplated that more or less items may be held in the storage container 2 in the event less or more days of are required to address the illness or injury. The storage container 2 provides the user with multiple, comprehensive items to address the illness or injury that would most likely not be purchased individually due to high over-the-counter costs and large package quantities that are often note utilized within a normal acute illness or injury course.

The storage container 2 described above provides the general public with cost-effective means to have access to multiple items to address minor acute illnesses and injuries. The average consumer will typically not pay the higher cost of buying all of the different over-the-counter items held within the storage container 2. The normal retail size and average consumer price to buy of the items contained within the storage container 2 would cost at least twice what the storage container 2 will sell for on the market. The storage container 2 also provides the general public with clear explanations and guidance to best utilize the items held within the storage container 2. The storage container 2 provides guidance to a comprehensive approach to utilize all of the modalities to address minor illness or injury contained within the storage container 2 to optimize the best health outcome. The average consumer does not typically know the specific over-the-counter products, items, and/or medications to buy and/or utilize during a minor acute illness or injury.

The storage container 2 is also configured to enhance the ability of the general public to take care of themselves during courses of minor acute illnesses and/or injuries. It is often difficult for the average consumer to know how to effectively address all of their symptoms during a minor acute illness or injury. It is also difficult to schedule an acute care appointment with a medical provider and, even if the medical provider is consulted, they do not have adequate time to address the effectiveness and detailed guidance for comprehensive items to address the illness or injury. The storage container 2 also provides the general public with comprehensive and convenient packaging to address minor acute illnesses and/or injuries. When a consumer is ill, they will often need quick and convenient access to address comprehensive symptom relief over the course of the illness. The storage container 2 provides confidence in one-stop-shopping to obtain multiple items for a designated condition. This convenience is also essential for periods of travel (hotels/foreign destinations), camping, hiking, or in situations where shopping time or selection may otherwise be severely limited.

While several aspects of the storage container for medication and medical items are shown in the accompanying figures and described in detail hereinabove, other aspects will be apparent to, and readily made by, those skilled in the art without departing from the scope and spirit of the disclosure. Accordingly, the foregoing description is intended to be illustrative rather than restrictive. The invention described hereinabove is defined by the appended claims and all changes to the invention that fall within the meaning and range of equivalency of the claims are to be embraced within their scope.

The invention claimed is:

1. A storage container for medication and medical items, comprising:
a first section defining a first cavity to receive a first portion of the medication and medical items;
a second section defining a second cavity to receive a second portion of the medication and medical items;
at least one tray positioned in at least one of the first section and the second section configured to hold the respective medication and medical items; and
at least one educational information card positioned in at least one of the first section and the second section, wherein the educational information card includes information pertaining to the medication and how to use the medical items held in the at least one tray.

2. The storage container of claim 1, wherein the at least one tray comprises:
a first tray and a second tray positioned in the first section and the second section, respectively, each configured to hold the respective medication and medical items of each tray.

3. The storage container of claim 1, wherein the at least one educational information card comprises:
a first educational information card and a second educational information card positioned in the first section and the second section, respectively, wherein each educational information card includes information pertaining to the respective medication and how to use the respective medical items held in each tray.

4. The storage container of claim 3, wherein each of the first educational information card and the second educational information card comprises:
a foldout including a plurality of educational information displays, wherein the foldout has a hinged connection with each tray.

5. The storage container of claim 3, wherein each of the first educational information card and the second educational information card is configured to form a lid for each of the first tray and the second tray, respectively.

6. The storage container of claim 1, wherein the at least one tray includes medication and medical items pertaining to a designated condition.

7. The storage container of claim 6, wherein the information pertaining to the medication and how to use the medical items pertaining to a designated condition includes condition-specific education configured to optimize a health outcome.

8. The storage container of claim 2, wherein the first tray and the second tray positioned in the first section and the second section, respectively, each includes medication and medical items pertaining to a designated condition.

9. The storage container of claim 8, wherein the information pertaining to the medication and how to use the medical items pertaining to a designated condition includes condition-specific education configured to optimize a health outcome.

10. The storage container of claim 1, wherein the first section and the second section are connected by a hinge and are configured to be fastened together by a fastening member.

11. The storage container of claim 1, further comprising:
an informational sleeve surrounding the storage container, wherein the informational sleeve includes information pertaining to the medication and how to use the medical items held in the storage container.

12. The storage container of claim 1, wherein the at least one educational information card includes at least one of: content pictures describing each medication and/or medical item, educational elements explaining reason for use for each medical item, and application education for each medication and/or how to use each medical item.

13. A storage container for medication and medical items, comprising:
a first section defining a cavity to receive a first portion of the medication and medical items;
a second section defining a second cavity to receive a second portion of the medication and medical items;
at least one divider positioned in at least one of the first section and the second section to create individual spaces to hold the medication and medical items; and
an educational information card positioned in the storage container, wherein the educational information card includes information pertaining to the medication and how to use the medical items held in the storage container.

14. The storage container of claim 13, wherein the storage container includes medication and medical items pertaining to a designated condition.

15. The storage container of claim 14, wherein the information pertaining to the medication and how to use the medical items pertaining to a designated condition includes condition-specific education configured to optimize a health outcome.

16. The storage container of claim 13, further comprising:
at least one cover panel positioned over the at least one divider.

17. The storage container of claim 16, wherein the at least one cover panel comprises a first portion and a second portion, wherein the first portion and the second portion are removably connected to one another.

18. The storage container of claim 13, wherein the educational information card comprises a first side and a second side, wherein the first side and the second side of the educational information card includes information pertaining to the medication and how to use the medical items held in the first section and the second section, respectively, of the storage container.

19. The storage container of claim 13, wherein the educational information card is fixedly attached to the storage container.

20. The storage container of claim 13, wherein the educational information card is removably positioned within the storage container.
